(19) United States
(12) Patent Application Publication
(10) Pub. No.: US 2005/0283163 A1
(43) Pub. Date: Dec. 22, 2005

(54) Intraocular lens implanting instrument

(76) Inventors: Valdemar Portney, Tustin, CA (US); Alexandre Abramov, Kazam (RU); Rifat Gaptelhakov, Kazam (RU)

Correspondence Address: SHELDON & MAK, INC, 225 SOUTH LAKE AVENUE, 9TH FLOOR, PASADENA, CA, 91101, US

(21) Appl. No.: 11/144,847

(22) Filed: Jun. 2, 2005

Related U.S. Application Data
(60) Provisional application No. 60/577,429, filed on Jun. 4, 2004.

(51) Int. Cl. A61F 9/00

(52) U.S. Cl. 606/107

(57) ABSTRACT

An instrument for implanting an elastically deformable intraocular lens has a forceps jaw, a stationary member and operations structure. The operations structure includes members capable of enabling the sequential (i) placing the lens in the undeformed state on the stationary member, (ii) closing the forceps jaws to fold the lens into its folded state and forming a folding chamber with a nozzle at its end, and (iii) pushing of the lens in its folded state axially out of the folding chamber through the nozzle so as to allow the lens to expand from its folded state to its undeformed state.

### BACKGROUND OF THE INVENTION

1. Field of the Invention

The present invention relates generally to the field of ophthalmics, more particularly to intraocular lenses (IOLs), and still more particularly to instruments for implanting IOLs in eyes and specifically anterior chambers of eyes.

2. Background Discussion

Until relatively recently, IOLs for aphakic eyes were typically made from rigid polymethyl methacrylate (PMMA), a hard, biocompatible, plastic material. Within the past few years, however, the manufacture of IOL's has largely shifted from rigid PMMA to soft, elastically deformable silicone or acrylic material that enables insertion of folded (or otherwise dimensionally-reduced) IOLs through substantially smaller ocular incisions that those required for the implanting of rigid IOL's. Such smaller ocular incisions typically minimize patient trauma, reduce the risk of surgical complications and speed post-surgical recovery.

In addition to continuing interest in implanting IOL's in aphakic eyes (that is, eyes from which the natural lens has been removed because of disease or injury), attention has recently been given to the implanting of IOL's in otherwise healthy phakic eyes (that is, eyes from which the natural lens has not been removed) to correct such common vision problems as myopia, hyperopia, presbyopia and astigmatism.

This implanting of corrective IOLs in phakic eyes is an often-attractive alternative to the wearing of corrective spectacles or contact lenses, which limit certain activities and even certain professions, or having performed such ocular surgical procedures on the cornea as radial keratomy (RK), photo-radial keratectomy (PRK) or LASIK, which may not be desired by or contra-indicated for some individuals.

In fact, the implanting of corrective IOLs in phakic eyes to correct vision problems is considered by many in the field of ophthalmics to be one of the remaining frontiers of vision correction.

Although aphakic IOL's are almost always implanted in the posterior chamber of the eye from which the natural lens has been removed, corrective IOL's for phakic eyes are usually implanted in the anterior chamber of the eye, between the cornea and the iris.

The small anterior chamber axial dimension, typically only about 3 mm between the posterior (rear) surface of the cornea and the anterior (front) surface of the crystalline lens, requires that anterior chamber IOL's typically be very thin to avoid undesirable contact with the easily-damaged endothelial layer of the cornea or anterior surface of the crystalline lens.

Elastically deformable IOL's are introduced into the eye, in this case, the anterior chamber of the eye, through some type of small IOL injector in which the IOL's are folded or deformed to pass through and out of the injector nozzle tip after the nozzle has been inserted through a small ocular incision. As the deformed IOL'S are pushed out of the nozzle tip, they elastically unfold, typically, in an uncontrolled manner, to regain their original optical shape. However, such uncontrolled unfolding of an IOL in the anterior chamber creates a serious risk of the IOL contacting and injuring the sensitive endothelial surface of the cornea or crystalline lens surface, thereby possibly causing a new vision problem.

Accordingly, a principal objective of the present invention is to provide an IOL implanting (insertion) instrument that allows precise loading of the IOL at a forceps type construction with a pusher of the lens located at the edge of the lens to avoid haptic deformation, then controlled folding of the IOL by forceps jaws forming the folding chamber with a nozzle at its end and then injecting the lens through the formed nozzle into the anterior chamber of the eye. It will be appreciated, however, that the IOL implanting instrument can also be used for the implanting of an elastically deformable IOL in the posterior chamber of an eye.

### SUMMARY

In accordance with the present invention, there is provided an instrument for implanting an elastically deformable intraocular lens in an eye. The IOL implanting instrument comprises forceps having two jaws, a stationary member located between the edges of the jaws and a pushing element located next to the intended location of the lens optic edge. Further included are operating means for enabling the sequential (i) placing the IOL in its undeformed state on the stationary member between the forceps jaws, (ii) closing the forceps jaws to fold the IOL and forming the folding chamber with the nozzle at its end, and (iii) pushing of the elastically deformed IOL axially out of the nozzle insertion end region for expanding into its undeformed state within the eye.

In accordance with a preferred embodiment of the invention, an instrument for implanting an elastically foldable IOL lens in an eye comprises a stationary element located between the edges of forceps jaws that can be opened for IOL loading and closed to folding the elastically deformable IOL, and a pushing element located at the optic edge. Further comprising the instrument are operating means for enabling the sequential (i) placing the IOL in its undeformed state on the stationary member with the pushing element located at the edge of the optic, (ii) closing the forceps jaws to fold the IOL and forming a folding chamber with a nozzle at its end, and (iii) pushing the elastically deformed IOL lens axially out of the nozzle insertion end region for expanding into its undeformed state within an eye.

A variation of the present IOL implanting instrument comprises rotating forceps jaws, which, when rotated to their closed condition form an IOL folding chamber, and IOL holding forceps that capture the IOL off an IOL cartridge. The holding forceps comprise a stationary jaw that slides under the IOL to take it off the cartridge and a pushing jaw that, together with the stationary jaw, holds the IOL for folding by the closing jaws of the rotating forceps and then pushes the IOL out of the IOL folding chamber (formed by the closed rotating forceps jaws) for unfolding of the IOL, for example, in a patient's eye.

### BRIEF DESCRIPTION OF THE DRAWINGS

The present invention can be more readily understood by a consideration of the following detailed description when taken in conjunction with the accompanying drawings, in which:

FIG. 1 is a drawing of the implanting instrument with its forceps jaw open and an IOL loaded in its undeformed state between the jaws against the stationary member with the pusher located next to optic edge;

FIG. 2 is a front view looking along line 2-2 of FIG. 1, showing the IOL in its unfolded state placed against A stationary member located between the edges of the forceps jaws;

FIG. 3 is a drawing of the implanting instrument with its forceps jaw closed to fold the IOL, and showing a side sliding knob locking the forceps jaws in their closed state;

FIG. 4 is a front view looking along line 4-4 of FIG. 3, showing the IOL in its folded state inside the closed forceps jaws and against the stationary member located between the edges of the forceps jaws;

FIG. 5 is a longitudinal cross-section of the front part of the instrument where the IOL is located inside the closed forceps jaws with the pushing element placed against the optic edge;

FIG. 6 is a drawing of the implanting instrument with its forceps jaw closed and the IOL being pushed out axially of the nozzle insertion end region and expanding into its original undeformed state;

FIG. 7 is a close-up view of the lens and front part of the implanting instrument of the FIG. 6 showing the pushing element located adjacent the optic edge;

FIG. 8 is a front view looking along line 8-8 of FIG. 7, showing the IOL injected from the implanting instrument by the pushing element and taking its original unfolded state:

FIG. 9 is a front view, similar to FIG. 2, of a variation IOL implanting instrument, showing an IOL in its normal, unfolded state between rotating forceps jaws shown in their open condition, the IOL being shown lifted off an IOL cartridge by a stationary jaw of holding forceps;

FIG. 10 is a front view similar to FIG. 9, showing the IOL being held by both jaws of the holding forceps, with a pushing jaw of the holding forceps on top of the IOL and the stationary jaw of the holding forceps underneath the IOL, and showing the IOL lifted upwardly into contact with the rotating forceps jaws which are shown still in their open condition;

FIG. 11 is a front view similar to FIG. 10, showing the IOL folded by the rotating forceps jaws shown rotated into their closed condition, the closed jaws of the rotating forceps forming an IOL folding chamber, the IOL still being shown held by the holding forceps; and

FIG. 12 is a front view similar to FIG. 11, showing a pushing element on the pushing jaw of the holding forceps pushing against an edge of the IOL optic to push the IOL off the stationary jaw of the holding forceps and out of the folding chamber formed by the closed rotating forceps jaws, thereby enabling the IOL to return to its normal, unfolded state.

In the various FIGS. the same elements and features are given the same reference numbers.

### DETAILED DESCRIPTION

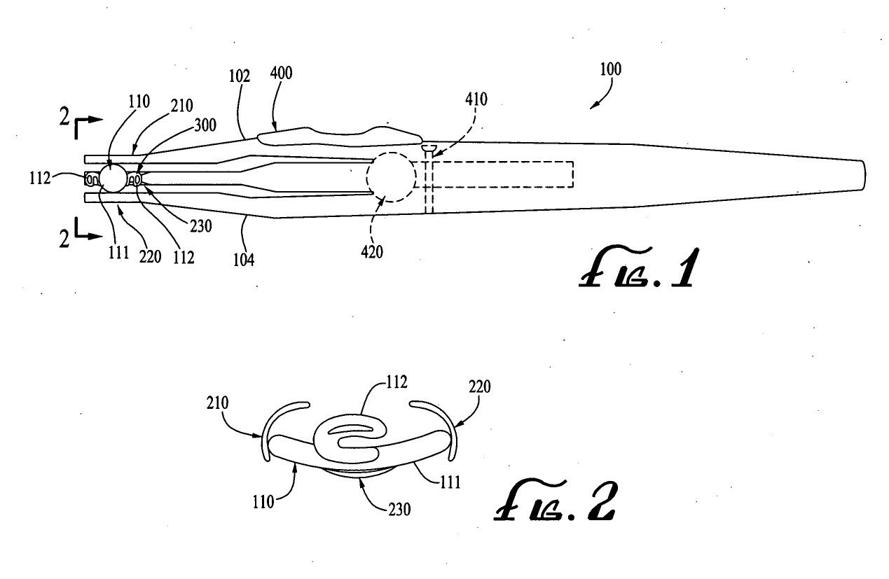

There is shown in FIG. 1 an implanting instrument 100 for an intraocular lens (IOL) 110 in accordance with a preferred embodiment of the present invention. Shown comprising instrument 100 are elongate forceps legs 102 and 104 having associated first and second forceps jaws 210 and 220, respectively, for holding and folding IOL 110. Included in instrument 100 are an elongate IOL supporting member 230 disposed between arcuate forceps jaws 210 and 220 beneath IOL 110 in its normal, undeformed state (FIG. 2) and an elongate IOL optic pushing member 300 controlled by a pusher button 420 (shown in broken lines). A forceps actuating lever 400, having an associated locking pin 410 for locking the forceps in its closed condition.

As shown in FIG. 2, IOL 110, comprising an optic 111 and opposing attachment elements (haptics) 112, is positioned in its normal, undeformed (unfolded) condition between forceps jaws 210 and 220 and resting on supporting member 230.

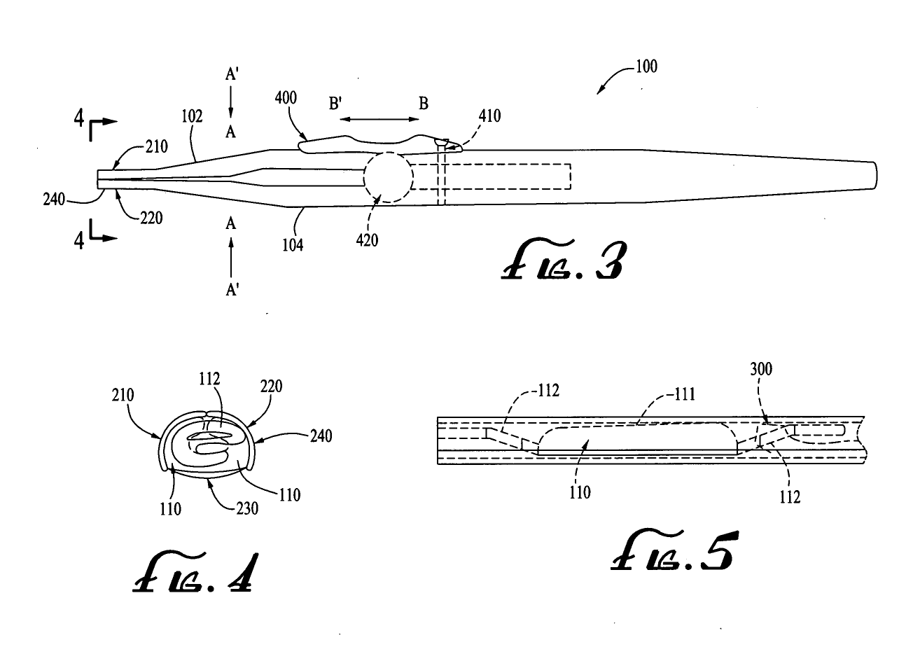

In FIG. 3 forceps legs 102 and 104 are shown closed by actuating lever 410 (which is locked in such condition (by locking pin 410) with IOL 110 folded between closed forceps jaws 210 and 220, while held in place by support member 230 (FIG. 4). It can also be seen from FIG. 4 that, in combination, closed forceps jaws 210 and 220 and support member 230 form a slender IOL insertion nozzle 240.

FIG. 5 depicts the manner in which a tip region of optic pushing member 300 is positioned for pushing against optic 111 of IOL 110 for pushing the deformed IOL out of the closed forceps jaws 210 and 220 (that is, out of nozzle 240), for example into a patient's eye (not shown).

In FIG. 6, with implanting instrument 100 viewed from its under side, pushing member 300 is shown advanced by pusher button 420 through closed forceps jaws 210 and 220 so as to push IOL 100 from the jaws and out of implanting instrument nozzle 240, for example into patient's eye (not shown), released IOL optic 111 being shown returned to its initial, undeformed condition. Forceps jaws 210 and 220 being held in their closed condition by locking pin 410 which locks actuating lever 400.

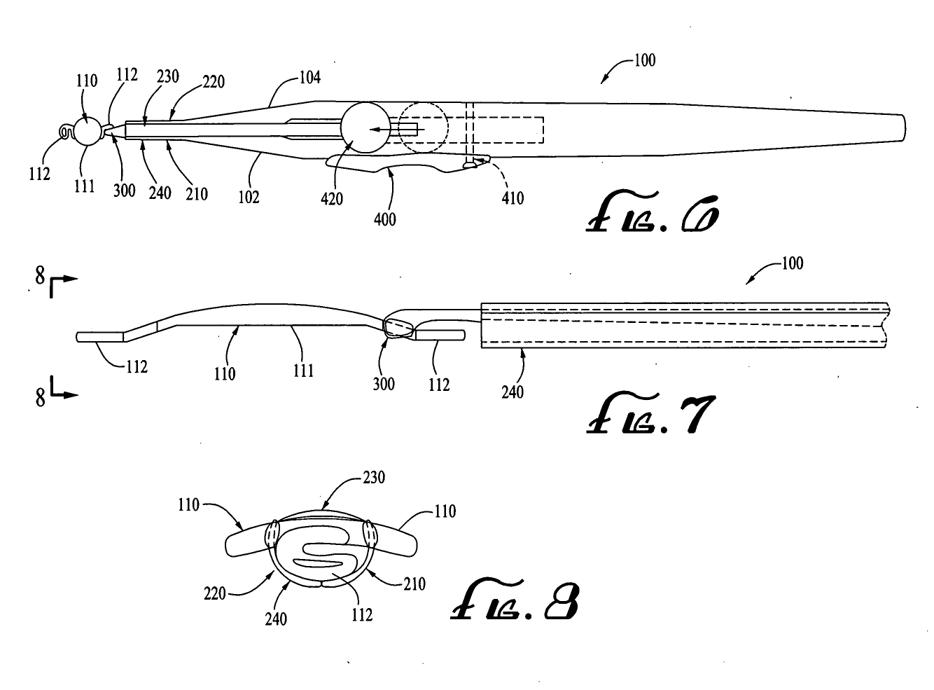

FIG. 7 illustrates the manner in which the pushing tip of pushing member engages optic 111 of IOL 110 while avoiding possibly damaging engagement with IOL attachment elements 112. FIG. 8 depicts IOL 110 released from forceps jaws 210 and 220 and, hence, nozzle 240, with IOL optic 111 unfolded to its original undeformed condition.

There is shown in FIGS. 9-12 front views of a variation IOL implanting instrument identified generally by reference number 100a. Shown in FIG. 9 are opposing arcuate jaws 310 and 320 attached to associated longitudinal, counter-rotating pivot shafts 330 and 340, In combination, jaws 310, 320 and pivot shafts 330, 340 form rotating (that is, pivoting) forceps identified generally by reference number 344. A lower, stationary (that is, longitudinally fixed) jaw 350 and an upper, pushing jaw 360, having an IOL pushing tab 370, comprise a holding forceps identified generally by reference number 374. As shown, IOL 110 is supported on lower jaw 350, which may also function as a scoop for lifting the IOL off a conventional IOL cartridge or carrier (not shown), and is positioned between rotating jaws 310 and 320, which are shown in their open condition, and below upper jaw 360. Reference number 380 identifies an internal region of instrument 100a in which holding forceps 374 are installed.

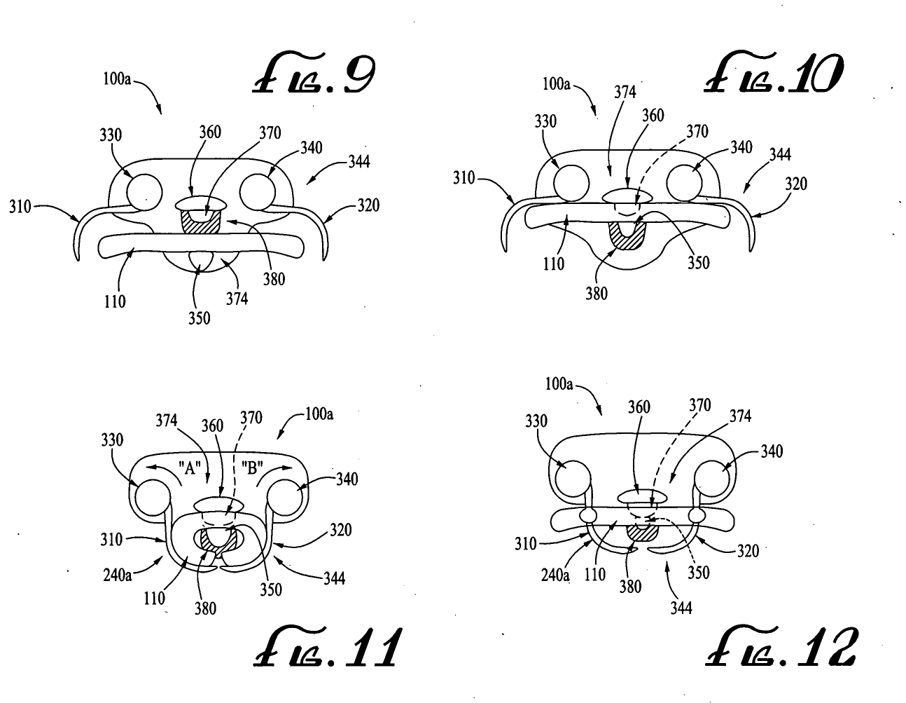

FIG. 10 depicts the next-in-sequence operational step in which IOL 110 has been elevated by lower jaw 350 into engagement with upper jaw 360, the IOL being held between lower and upper jaws. IOL 110 is now also in contact with rotating jaws 310 and 320, which are still in their un-rotated, open condition.

FIG. 11 depicts the next-in-sequence operational step in which IOL 110, still held between lower and upper jaws 350 and 360, is compressed into its folded condition by rotation of jaw 310 in a counterclockwise direction (arrow “A”) and the simultaneous, rotation of opposing jaw 320 in a clockwise direction. Such simultaneous counter rotation of jaws 310 and 320 into their closed condition depicted can be accomplished by gearing shafts 330 and 340 together, in a well known manner (not shown). At this point, closed rotating jaws 310 and 320 with upper jaw 360 form an IOL implanting or injection nozzle 240a.

FIG. 12 depicts the next-in-sequence operational step in which IOL 110 is pushed outwardly from between closed rotating jaws 310 and 320 and from between lower and upper jaws 350 and 360 (that is, out of nozzle 240a) by pushing element 370 of the upper jaw which pushes against an edge of the IOL optic. As a result, IOL 110 is enabled to expand from its folded condition (FIG. 11) back into its normal, unfolded condition (FIG. 12), for example, in a patient's eye (not shown).

All above-described parts of implanting instruments 100 and 100a are preferably formed from a medical grade of stainless steel (or other biocompatible materials) so that the complete instrument can be sterilized, for example, by autoclaving, after each patient use.

Although there are described and illustrated herein an instrument for implanting IOLs in patients' eyes and a variation thereof, for purposes of illustrating the manner in which the present invention may be used to advantage, it is to be understood that the invention is not limited thereto. Consequently, any and all variations and equivalent arrangements which may occur to those skilled in the applicable art are to be considered to be within the scope and spirit of the invention as set forth in the claims which are appended hereto as part of this application.

1. An instrument for implanting an elastically deformable intraocular lens in an eye, the intraocular lens being capable of being alternatively disposed in a folded state and in an undeformed state, said instrument comprising:
(a) forceps jaws defining an open position and a closed position;
(b) a stationary member; and
(c) operating means for enabling the sequential
(i) placing the lens in the undeformed state on the stationary member,
(ii) closing the forceps jaws to fold the lens into its folded state and forming a folding chamber with a nozzle at its end, and
(iii) pushing of the lens in its folded state axially out of the folding chamber through the nozzle so as to allow the lens to expand from its folded state to its undeformed state.

2. The instrument of claim 1 wherein the forceps jaws are arcuate.

3. The instrument of claim 1 wherein the forceps jaws consist of a pair of forceps jaws attached to forceps legs which are supported by a supporting member.

4. The instrument of claim 1 wherein the forceps jaws are opened and closed by an actuating lever.

5. The instrument of claim 4 wherein the forceps jaws are locked in the closed position by a locking pin.

6. The instrument of claim 1 wherein the operating means comprises a pushing member.

7. The instrument of claim 6 wherein the pushing member is operated by a pusher button.

8. The instrument of claim 1 wherein the forceps jaws are rotatable.

9. The instrument of claim 8 wherein the forceps jaws are rotatable via counter rotating pivot shafts.

10. The instrument of claim 8 wherein the forceps jaws comprise a pair of forceps jaws, a stationary jaw and a pushing jaw.

11. An instrument for implanting an elastically deformable intraocular lens in an eye, said instrument comprising:
(a) forceps jaws;
(b) a stationary member; and
(c) operating means for enabling the sequential
(i) placing the lens in the undeformed state on the stationary member with the pushing element located at an edge of the optic, and
(ii) closing the forceps jaws to fold the lens and form a-folding chamber with the nozzle at its end, and
(iii) pushing of an elastically deformed intraocular lens axially out of the formed nozzle insertion end region for expanding into its undeformed shape.
